SEQUENCE LISTING (11 sequences)

SEQ ID NO: 1 - protein, 68 aa, Artificial sequence
  Synthetic Human RANTES polypeptide
SPYSSDTTPCCFAYIARPLPRAHIKEYFYTSGKCSNPAVVFVTRKNRQVCANPEKKWVREYINSLEMS

SEQ ID NO: 2 - protein, 68 aa, Artificial sequence
  Synthetic rhesus wild-type RANTES polypeptide
SPHASDTTPCCFAYIARPLPRAHIKEYFYTSGKCSNPAVVFVTRKNRQVCANPEKKWVREYINSLEMS

SEQ ID NO: 3 - protein, 68 aa, Artificial sequence
  Synthetic rhesus C1C5 mutant RANTES polypeptide
CPHACDTTPCCFAYIARPLPRAHIKEYFYTSGKCSNPAVVFVTRKNRQVCANPEKKWVREYINSLEMS

SEQ ID NO: 4 - DNA, 109 bp, Artificial sequence
  Synthetic Wild-type rhesus forward primer
cttagcggccgccaccatgtggtggcgcctgtggtggctgctgctgctgctgctgctgctgtggcccatggtgtgggcctccccacacgcctccgacaccacaccctgc

SEQ ID NO: 5 - DNA, 110 bp, Artificial sequence
  Synthetic rhesus C1C5 mutant forward primer
cttagcggccgccaccatgtggtggcgcctgtggtggctgctgctgctgctgctgctgctgtggcccatggtgtgggcctgcccacacgcctgcgacaccacaccctgct

SEQ ID NO: 6 - DNA, 37 bp, Artificial sequence
  Synthetic reverse rhesus primer
cttagcggccgctcagctcatctccaaagagttgatg

SEQ ID NO: 7 - DNA, 194 bp, Artificial sequence
  Synthetic T-20 oligonucleotide
gcggccgccaccatgtggtggcgcctgtggtggctgctgctgctgctgctgctgctgtggcccatggtgtgggcctacacctccctgatccactccctgatcgaggagtcccagaaccagcaggagaagaacgagcaggagctgctggagctggacaagtgggcctccctgtggaactggttctgagcggccgc

SEQ ID NO: 8 - DNA, 110 bp, Artificial sequence
  Synthetic artificial signal peptide primer
attcagcggccgccaccatgtggtggcgcctgtggtggctgctgctgctgctgctgctgctgtggcccatggtgtgggccatggagtgggacagggagatcaacaactac

SEQ ID NO: 9 - DNA, 107 bp, Artificial sequence
  Synthetic systatin leader peptide primer
attcagcggccgccaccatggcccgccccctgtgcaccctgctgctgctgatggccaccctggccggcgccctggccatggagtgggacagggagatcaacaactac

SEQ ID NO: 10 - DNA, 119 bp, Artificial sequence
  Synthetic A1AT leader peptide primer
attcagcggccgccaccatgccctcctccgtgtcctggggcatcctgctgctggccggcctgtgctgcctggtgcctgtgtccctggccatggagtgggacagggagatcaacaactac

SEQ ID NO: 11 - DNA, 39 bp, Artificial sequence
  Synthetic reverse T-1249 primer
attcagcggccgcctcaccacagggaggcccacttgtcc

(19) United States
(12) Patent Application Publication
(10) Pub. No.: US 2009/0169513 A1
(43) Pub. Date: Jul. 2, 2009

(54) Expression of virus entry inhibitors and recombinant AAV therefor

(75) Inventors: Philip R. Johnson, JR., Columbus, OH (US); Kelly Reed Clark, Columbus, OH (US)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: NATIONWIDE CHILDREN'S HOSPITAL, Columbus, OH (US)

(21) Appl. No.: 12/079,002

(22) Filed: Mar. 24, 2008

Related U.S. Application Data
(63) Continuation of application No. 11/319,954, filed on Dec. 28, 2005, now abandoned.
(60) Provisional application No. 60/640,113, filed on Dec. 29, 2004.

(51) Int. Cl. A61K 45/00; C07H 21/04; C12N 7/01; C12P 21/02; A61P 43/00

(52) U.S. Cl. 424/93.2; 536/23.72; 435/235.1; 435/69.1

(57) ABSTRACT

The present invention relates generally to the use of recombinant adeno-associated viruses (rAAV) for gene delivery and more specifically to the use of rAAV to deliver genes encoding human immunodeficiency virus entry inhibitors to target cells in mammals.

### FIELD OF INVENTION

The present invention relates generally to the use of recombinant adeno-associated viruses (rAAV) for gene delivery and specifically to the use of rAAV to deliver DNA encoding, and direct expression of, virus entry inhibitors in target cells in mammals. More particularly, the invention relates to the use of rAAV to deliver and direct expression of DNA encoding human immunodeficiency virus entry inhibitors.

### BACKGROUND

Adeno-associated virus (AAV) is a replication-deficient parvovirus, the single-stranded DNA genome of which is about 4.7 kb in length including 145 nucleotide inverted terminal repeat (ITRs). The nucleotide sequence of the AAV serotype 2 (AAV2) genome is presented in Srivastava et al., J. Virol., 45: 555-564 (1983) as corrected by Ruffing et al., J. Gen. Virol., 75: 3385-3392 (1994). Cis-acting sequences directing viral DNA replication (rep), encapsidation/packaging and host cell chromosome integration are contained within the ITRs. Three AAV promoters, p5, p19, and p40 (named for their relative map locations), drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40)from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

When wild type AAV infects a human cell, the viral genome can integrate into chromosome 19 resulting in latent infection of the cell. Production of infectious virus does not occur unless the cell is infected with a helper virus (for example, adenovirus or herpesvirus). In the case of adenovirus, genes E1A, E1B, E2A, E4 and VA provide helper functions. Upon infection with a helper virus, the AAV provirus is rescued and amplified, and both AAV and adenovirus are produced.

AAV possesses unique features that make it attractive as a vaccine vector for expressing irmnunogenic peptides/polypeptides and as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA such as a gene cassette containing a promoter, a DNA of interest and a polyadenylation signal. The rep and cap proteins may be provided in trans. Another significant feature of AAV is that it is an extremely stable and hearty virus. It easily withstands the conditions used to inactivate adenovirus (56° to 65° C. for several hours), making cold preservation of rAAV vectors less critical. AAV may even be lyophilized. Finally, AAV-infected cells are not resistant to superinfection.

Multiple studies have demonstrated long-term (>1.5 years) rAAV mediated protein expression in muscle. See, Clark et al., Hum. Gene Ther., 8: 659-669 (1997); Kessler et al., Proc. Natl. Acad. Sci. USA, 93: 14082-14087 (1996); and Xiao et al., J. Virol., 70: 8098-8108 (1996). See also, Chao et al., Mol. Ther., 2:619-623 (2000) and Chao et al., Mol. Ther., 4:217-222 (2001). Moreover, because muscle is highly vascularized, rAAV transduction has resulted in the appearance of transgene products into the systemic circulation following intramuscular injection as described in Herzog et al., Proc. Natl. Acad. Sci. USA, 94: 5804-5809 (1997) and Murphy et al., Proc. Natl. Acad. Sci. USA, 94: 13921-13926 (1997). Moreover, Lewis et al., J. Virol., 76: 8769-8775 (2002) demonstrated that skeletal myofibers possess the necessary cellular factors for correct antibody glycosylation, folding, and secretion, indicating that muscle is capable of stable expression of secreted protein therapeutics.

HIV-1 is considered to be the causative agent of Acquired Immunodeficiency Syndrome (AIDS) in the United States. As assessed by the World Health Organization, more than 40 million people are currently infected with HIV and 20 million people have already perished from AIDS. Thus, HIV infection is considered a worldwide pandemic.

There are two currently recognized strains of HIV, HIV-1 and HIV-2. HIV-1 is the principal cause of AIDS around the world. HIV-1 has been classified based on genomic sequence variation into clades. For example, Clade B is the most predominant in North America, Europe, parts of South America and India; Clade C is most predominant in Sub-Saharan Africa; and Clade E is most predominant in southeastern Asia. HIV-1 infection occurs primarily through sexual transmission, transmission from mother to child or exposure to contaminated blood or blood products.

HIV-1 consists of a lipid envelope surrounding viral structural proteins and an inner core of enzymes and proteins required for viral replication and a genome of two identical linear RNAs. In the lipid envelope, viral glycoprotein 41 (gp 41)anchors another viral envelope glycoprotein 120 (gp 120) that extends from the virus surface and interacts with receptors on the surface of susceptible cells. The HIV-1 genome is approximately 10,000 nucleotides in size and comprises nine genes. It includes three genes common to all retroviruses, the gag, pol and env genes. The gag gene encodes the core structural proteins, the env gene encodes the gp120 and gp41 envelope proteins, and the pol gene encodes the viral enzymes reverse transcriptase (RT), integrase and protease (pro). The genome comprises two other genes essential for viral replication, the tat gene encoding a viral promoter transactivator and the rev gene which also facilitates gene transcription. Finally, the nef, vpu, vpr, and vif genes are unique to lentiviruses and encode polypeptides the functions of which are described in Trono, Cell, 82: 189-192 (1995).

The process by which HIV-1 infects human cells involves interaction of proteins on the surface of the virus with proteins on the surface of the cells. The common understanding is that the first step in HIV infection is the binding of HIV-1 glycoprotein (gp) 120 to cellular CD4 protein. This interaction causes the viral gp120 to undergo a conformational change and bind to other cell surface proteins, such as CCR5 or CXCR4 proteins, allowing subsequent fusion of the virus with the. cell. CD4 has thus been described as the primary receptor for HIV-1 while the other cell surface proteins are described as coreceptors for HIV-1.

HIV-1 infection is characterized by an asymptomatic period between infection with the virus and the development of AIDS. The rate of progression to AIDS varies among infected individuals. AIDS develops as CD4-positive cells, such as helper T cells and monocytes/macrophages, are infected and depleted. AIDS is manifested as opportunistic infections, increased risk of malignancies and other conditions typical of defects in cell-mediated immunity. The Centers for Disease Control and Prevention clinical categories of pediatric, adolescent and adult disease are set out in Table I of Sleasman and Goodenow, J. Allergy Clin. Immunol., 111(2): S582-S592 (2003).

Predicting the likelihood of progression to AIDS involves monitoring viral loads (viral replication) and measuring CD4-positive T cells in infected individuals. The higher the viral loads, the more likely a person is to develop AIDS. The lower the CD4-positive T cell count, the more likely a person is to develop AIDS.

At present, antiretroviral drug therapy (ART) is the only means of treating HIV infection or preventing HIV-1 transmission from one person to another. At best, even with ART, HIV-1 infection is a chronic condition that requires lifelong drug therapy and there can still be a slow progression to disease. ART does not eradicate HIV-1 because the virus can persist in latent reservoirs. Moreover, treatment regimens can be toxic and multiple drugs must be used daily. There thus is an urgent need to develop effective vaccines and treatments for HIV-1 infection.

### SUMMARY OF INVENTION

The present invention exploits the unique gene-delivery properties of AAV to deliver and direct expression of proteins (other than antibodies) that inhibit viruses. The vectors are contemplated for use in preventing viral infection and in treating viral infection, particularly HIV infection.

In a first aspect, the invention provides rAAV genomes. The rAAV genomes comprise AAV ITRs flanking a gene cassette of DNA encoding one or more virus entry inhibitor proteins operatively linked to transcriptional control DNA, specifically promoter DNA and polyadenylation signal sequence DNA, functional in target cells. The gene cassette may also include intron sequences to facilitate processing of the RNA transcript when expressed in mammalian cells. The rAAV genomes of the invention lack AAV rep and cap DNA. AAV DNA in the rAAV genomes may be from any AAV serotype for which a recombinant virus can be derived.

Proteins that are virus entry inhibitors according to the invention may be peptides or polypeptides. The proteins may inhibit virus entry into host target cells by binding to the virus or by binding to the host target cell. Examples of HIV virus entry inhibitors that bind to HIV include, but are not limited to, peptides T20 (also known as DP178) [Wild et al., Proc. Nat'l. Acad. Sci. USA, 91:9770-9774 (1994)], T1249 [Kilby et al., N. Engl. J. Med., 348:2228-2238 (2003)], C34 [Rimsky et al., J. Virol., 72:986-993 (1998)], T649 (Rimsky et al., supra) and 5-helix [Root et al., Science, 291:884-888 (2001)] that inhibit virus:cell fusion and CD4, CCR5, CXCR4 cellular receptors or portions thereof that bind HIV. Examples of HIV virus entry inhibitors that bind to host target cells include, but are not limited to, chemokines RANTES [Polo et al., Eur. J. Immunol., 30:3190-3198 (2000)] and SDF-1 [Berger et al., Annu. Rev. Immunol., 17:657-700 (1999)].

Proteins that are virus entry inhibitors according to the invention may be chimeric (i.e., fusion) proteins. Chimeric virus entry inhibitor proteins may exhibit enhanced secretion and/or stability. For example, peptides like T20 may be fused to native molecules like human alpha-1-antitrypsin. Chimeric virus entry inhibitor proteins may comprise multiple virus entry inhibitor proteins. For example, a peptide like T20 may be fused to the N-terminus of human alpha-1-antitrypsin while a chemokine like RANTES may be fused to the C-terminus.

The invention contemplates rAAV genomes that express one or more proteins that inhibit virus entry including, but not limited to, entry of HIV, Hepatitis B virus, Hepatitis C virus, Epstein Barr Virus, influenza virus and Respiratory Syncytial Virus.

In another aspect, the invention provides DNA vectors comprising rAAV genomes of the invention. The vectors are transferred to cells permissible for infection with a helper virus of AAV (e.g., adenovirus, E1-deleted adenovirus or herpesvirus) for assembly of the rAAV genome into infectious viral particles. Techniques to produce rAAV particles, in which a AAV genome to be packaged, rep and cap genes, and helper virus functions are provided to a cell are standard in the art. Production of rAAV requires that the following components are present within a single cell (denoted herein as a packaging cell): a rAAV genome, AAV rep and cap genes separate from the rAAV genome, and helper virus functions. The AAV rep and cap genes may be from any AAV serotype for which recombinant virus can be derived and may be from a different AAV serotype than the rAAV genome ITRs.

A method of generating a packaging cell is to create a cell line that stably expresses all the necessary components for AAV particle production. For example, a plasmid (or multiple plasmids) comprising a rAAV genome, AAV rep and cap genes separate from the rAAV genome, and a selectable marker, such as a neomycin resistance gene, are integrated into the genome of a cell. The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

The invention thus provides packaging cells that produce infectious rAAV. In one embodiment packaging cells may be stably transformed cancer cells such as HeLa cells, 293 cells and PerC.6 cells (a cognate 293 line). In another embodiment, packaging cells are cells that are not transformed cancer cells such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

In another aspect, the invention provides rAAV (i.e., infectious encapsidated rAAV particles) comprising a rAAV genome of the invention. Embodiments include, but are not limited to, the following exemplified rAAV₁/CMV/T20, rAAV₁/CMV/T-1249, rAAV₁/CMV/RANTES, rAAV₁/CMV/rhRANTES(wt) and rAAV₁/CMV/mRANTES (C1C5). The vector nomenclature is the rAAV serotype/promoter element/virus inhibitor protein. The rAAV may be purified by methods standard in the art such as by column chromatography or cesium chloride gradients.

In another embodiment, the invention contemplates compositions comprising rAAV of the present invention. These compositions may be used to treat and/or prevent viral infections (acute and chronic viral infections) in particular AIDS. In one embodiment, compositions of the invention comprise a rAAV encoding a virus entry inhibitor protein of interest. In other embodiments, compositions of the present invention may include two or more rAAV encoding different viral entry inhibitor proteins (including chimeric proteins) of interest. In particular for neutralizing HIV-1, administration of a rAAV mixture which results in expression of several HIV entry inhibitor proteins, or a mixture of inhibitors that inhibit different steps in the HIV infection cycle, may increase neutralization of the virus. Administration may precede, accompany or follow ART.

Compositions of the invention comprise rAAV in a pharmaceutically acceptable carrier. The compositions may also comprise other ingredients such as diluents and adjuvants. Acceptable carriers, diluents and adjuvants are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics or polyethylene glycol (PEG).

Titers of rAAV to be administered in methods of the invention will vary depending, for example, on the particular rAAV, the mode of administration, the treatment goal, the individual, and the cell type(s) being targeted, and may be determined by methods standard in the art.

Methods of transducing a target cell with rAAV, in vivo or in vitro, are contemplated by the invention. The in vivo methods comprise the step of administering an effective dose or doses of a composition comprising a rAAV of the invention to an animal (including a human being) in need thereof. If the dose is administered prior to infection by a virus, the administration is prophylactic. If the dose is administered after infection by a virus, the administration is therapeutic. An effective dose is a dose sufficient to alleviate (eliminate or reduce) at least one symptom associated with the infection or disease state being treated. In one embodiment, alleviation of symptoms prevents progression of a viral infection to a disease state. In another embodiment, alleviation of symptoms prevents progression to, or progression of, a disease state caused by a viral infection. Viral infections (including acute and chronic viral infections) to be treated include, but are not limited to, HIV-1 infection, Hepatitis B virus infection, Hepatitis C virus infection, Epstein Barr Virus infection, influenza infection and Respiratory Syncytial Virus infection. Administration of an effective dose of the compositions may be by routes standard in the art including, but not limited to, intramuscular, parenteral, intravenous, oral, buccal, nasal, pulmonary, intracranial, intraosseous, intraocular, rectal, or vaginal. Route(s) of administration and serotype(s) of AAV components of rAAV (in particular, the AAV ITRs and capsid protein) of the invention may be chosen and/or matched by those skilled in the art taking into account the infection and/or disease state being treated and the target cells/tissue(s) that are to express the virus entry inhibitor protein(s).

In particular, actual administration of rAAV of the present invention may be accomplished by using any physical method that will transport the rAAV recombinant vector into the target tissue of an animal. Administration according to the invention includes, but is not limited to, injection into muscle, the bloodstream and/or directly into the liver. Simply resuspending a rAAV in phosphate buffered saline has been demonstrated to be sufficient to provide a vehicle useful for muscle tissue expression, and there are no known restrictions on the carriers or other components that can be co-administered with the vector (although compositions that degrade DNA should be avoided in the normal manner with vectors). Capsid proteins of a rAAV may be modified so that the rAAV is targeted to a particular target tissue of interest such as muscle. Pharmaceutical compositions can be prepared as injectable formulations or as topical formulations to be delivered to the muscles by transdermal transport. Numerous formulations for both intramuscular injection and transdermal transport have been previously developed and can be used in the practice of the invention. The rAAV can be used with any pharmaceutically acceptable carrier for ease of administration and handling.

For purposes of intramuscular injection, solutions in an adjuvant such as sesame or peanut oil or in aqueous propylene glycol can be employed, as well as sterile aqueous solutions. Such aqueous solutions can be buffered, if desired, and the liquid diluent first rendered isotonic with saline or glucose. Solutions of rAAV as a free acid (DNA contains acidic phosphate groups) or a pharmacologically acceptable salt can be prepared in water suitably mixed with a surfactant such as hydroxpropylcellulose. A dispersion of rAAV can also be prepared in glycerol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques well-known to those skilled in the art.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonger absorption of the injectable compositions can be brought about by use of agents delaying absorption, for example, aluminum monostearate and gelatin.

Sterile injectable solutions are prepared by incorporating rAAV in the required amount in the appropriate solvent with various of the other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique which yield a powder of the active ingredient plus any additional desired ingredient from the previously sterile-filtered solution thereof.

Transduction with rAAV can also be carried out in vitro. In one embodiment, desired target muscle cells are removed from the subject, transduced with rAAV and reintroduced into the subject. Alternatively, syngeneic or xenogeneic muscle cells can be used where those cells will not generate an inappropriate immune response in the subject.

Suitable methods for the transduction and reintroduction of transduced cells into a subject are known in the art. In one embodiment, cells can be transduced in vitro by combining rAAV with muscle cells, e.g., in appropriate media, and screening for those cells harboring the DNA of interest using conventional techniques such as Southern blots and/or PCR, or by using selectable markers. Transduced cells can then be formulated into pharmaceutical compositions, and the composition introduced into the subject by various techniques, such as by intramuscular, intravenous, subcutaneous and intraperitoneal injection, or by injection into smooth and cardiac muscle, using e.g., a catheter.

Transduction of cells with rAAV of the invention results in sustained expression of virus entry inhibitor proteins. The present invention thus provides methods of delivering rAAV which express virus entry inhibitor proteins to an animal, preferably a human being. These methods include transducing tissues (including but not limited to muscle, liver and brain) with one or more rAAV of the present invention. Transduction may be carried out with gene cassettes comprising tissue specific control elements. For example, one embodiment of the invention provides methods of transducing muscle cells and muscle tissues directed by muscle specific control elements, including, but not limited to, those derived from the actin and myosin gene families, such as from the myoD gene family (See: Weintraub et al., Science 251: 761-766, 1991), the myocyte-specific enhancer binding factor MEF-2 (Cserjesi and Olson, Mol. Cell Biol. 11: 4854-4862, 1991), control elements derived from the human skeletal actin gene (Muscat et al., Mol. Cell Biol. 7: 4089-4099, 1987), the cardiac actin gene, muscle creatine kinase sequence elements (See: Johnson et al. Mol. Cell Biol. 9:3393-3399, 1989) and the murine creatine kinase enhancer (mCK) element, control elements derived from the skeletal fast-twitch troponin C gene, the slow-twitch cardiac troponin C gene and the slow-twitch troponin I gene: hypozia-inducible nuclear factors (Semenza et al., Proc. Natl. Acad. Sci. USA 88: 5680-5684, 1991), steroid-inducible elements and promoters including the glucocorticoid response element (GRE) (See: Mader and White, Proc. Natl. Acad. Sci. USA 90: 5603-5607,1993), and other control elements.

Muscle tissue is a attractive target for in vivo gene delivery and gene therapy, because it is not a vital organ and is easy to access. rAAV based on alternate serotypes (e.g. AAV-1 [Xiao et al., J. Virol., 73(5): 3994-4003 (1999)] and AAV-5 [Chiorini et al., J. Virol., 73(2): 1309-1319 (1999)]) may transduce skeletal myocytes more efficiently than AAV-2. The invention contemplates sustained expression of biologically active virus entry inhibitor proteins from transduced myofibers.

By “muscle cell” or “muscle tissue” is meant a cell or group of cells derived from muscle of any kind, including skeletal muscle, smooth muscle, e.g. from the digestive tract, urinary bladder and blood vessels, cardiac, and excised from any area of the body. Such muscle cells may be differentiated or undifferentiated, such as myoblasts, myocytes, myotubes, cardiomyocytes and cardiomyoblast. Since muscle tissue is readily accessible to the circulatory system, a protein produced and secreted by muscle cells and tissue in vivo will logically enter the bloodstream for systemic delivery, thereby providing sustained, therapeutic levels of protein secretion from muscle.

The term “transduction” is used to refer to the delivery of entry inhibitor DNA to a recipient cell either in vivo or in vitro, via a replication-defificient rAAV of the invention resulting in expression of a functional virus entry inhibitor protein by the recipient cell.

Thus, the invention provides methods of administering an effective dose (or doses) of rAAV that encode proteins that inhibit virus entry to a patient in need thereof. Inhibition according to the invention is a reduction in infectivity of a primary viral isolate as measured by an in vitro or in vivo assay known in the art. Multiple assays are known in the art. Entry inhibitor-mediated neutralization of HIV-1 can be measured in an MT-2 cell-killing assay using Finter's neutral red to quantify viable cells [Montefiori et al., J. Clin. Microbiol., 26:231-235 (1988)]. An alternative cell-based HIV-1 infectivity assay was recently developed that utilizes single-cycle HIV-1 pseudovirion particles encoding the firefly luciferase report gene [Richman et al., Proc. Nat'l. Acad. Sci. USA, 100:4144-4149 (2003)]. Neutralization of the HIV-1 pseudovirion results in reduction of luciferase expression in the assay. The HIV-1 pseudovirion particles are readily pseudotyped with various CCR5, CXC4, or dual-tropic utilizing envelopes to determine neutralization efficacy and breadth.

Inhibition may result in clearance of a virus in the patient (i.e., sterilization) or may slow progression to a disease state caused by a virus. In one embodiment, methods of the invention include the administration of an effective dose (or doses) of rAAV of the invention encoding HIV-1 entry inhibitor protein(s) to prevent progression of a patient at risk for infection or infected with HIV-1 to AIDS. Preferred methods result in one or more of the following in the individual: a reduction of viral loads, maintenance of low viral loads, an increase in CD4-positive T cells, stabilization of CD4-positive T cells, reduced incidence or severity of opportunistic infections, reduced incidence of malignancies, and reduced incidence or severity of conditions typical of defects in cell-mediated immunity. The foregoing are each in comparison to an individual that, according to the art, has progressed or will likely progress to AIDS.

### BRIEF DESCRIPTION OF DRAWING

FIG. 1 depicts a RANTES sequence alignment.

FIG. 2 is a graph showing RANTES protein levels in cell culture supernatant.

FIG. 3 is a graph showing RANTES protein production from single-stranded and double-stranded production plasmids.

FIG. 4 is an autoradiograph of a Southern blot showing rAAV₁/rhRANTES replication intermediates.

FIG. 5 is a graph showing RANTES protein production in C12 cells.

FIG. 6 shows sequences of T-20 and T-1249 viral entry inhibitor proteins and where they bind to HIV-1 gp41.

FIG. 7 shows T-1249 production.

### DETAILED DESCRIPTION

The examples below describe two embodiments of stable delivery and expression of—HIV cell entry inhibitor proteins via viral gene transfer. The embodiments exploit the ability of rAAV to effect long-term delivery to, expression of genes in, and secretion of proteins from mature skeletal muscle. The goal of secretion of HIV-1 cell entry inhibitors into circulation is to inhibit HIV-1 replication. The examples illustrating embodiments of the invention include Example 1 describing use of RANTES chemokine derivatives to inhibit HIV-1 infection via CCR5 co-receptor blockade and Example 2 describing the delivery and expression of genes encoding HIV-1 fuision inhibitor peptides to inhibit HIV-1 replication and growth.

### EXAMPLE 1

### Use of Rantes Chemokine Derivatives to Inhibit HIV-1 Infection Via CCR5 Co-Receptor Blockade

Since primary HIV-1 isolates almost exclusively utilize CCR5 as the co-receptor for initial infection of cells, the chemokine RANTES (a natural CCR5 ligand) represents an ideal candidate for competitive blockade of the CCR5 co-receptor. The present inventors contemplate that elevated plasma levels of the RANTES chemokine will significantly attenuate or prevent HIV-1 infection of CD4+ cells and that the approach will be well-tolerated in vivo, since individuals who are deficient in CCR5 signaling are healthy and lack obvious immunological defects.

As described below, rhesus RANTES genes (wild-type and a non-signaling mutant) have been cloned into rAAV-1 vectors and are delivered into mouse muscle tissue. In order to maximize circulating rhRANTES expression levels as well as decease its proinflammatory activities, optimized molecular constructs were generated. First, an optimized leader sequence was added onto the N-terminus of rhRANTES to increase the efficiency of protein secretion from muscle cells into the systemic circulation. Second, a mutant rhRANTES (C1 C5) was constructed that retains the ability to associate with the CCR5 co-receptor but lacks chemotactic properties to minimize the potential for undesirable inflammatory responses imparted by RANTES overexpression on the cell-signaling cascade in vivo. C1C5 has two Ser→Cys substitutions at positions 1 and 5. Polo et al., Eur. J. Immunol., 30: 3190-3198 (2000) demonstrated that this mutated form of RANTES (C1C5) has a reduced ability to induce chemotaxis, but increased HIV-1 blocking activity when compared to wild-type RANTES. Third, to maximize gene transfer levels in muscle rAAV-1 serotype rather than AAV-2 serotype vectors were constructed. Fourth, to enhance the specific activity (potency) of the rAAV/chemokine vectors, self-complementary rAAV/chemokine vectors were constructed. McCarty et al., Gene Ther., 10: 2112-2118 (2003) showed that hairpin vectors rapidly form transcriptionally active double-stranded templates within a transduced cell, resulting in increased expression levels (typically 10-fold) and expression kinetics compared to standard single-strand rAAV vectors.

A. Amplification of rhRANTES DNA

Macaca mulatta (rhesus) specific RANTES PCR primers were designed and used to PCR amplify both wild-type and mutant forms of the rhesus RANTES from a plasmid encoding the human RANTES cDNA (pORF-hRANTES; InvivoGen Inc.). Forward primers for both wild-type and mutant forms were designed to contain an optimized synthetic leader sequence.

Wild-type rhesus forward primer:
5′CTTAGCGGCCGCCACCATGTGGTGGCGCCTGTGGTGGCTGCTGCTGCT
GCTGCTGCTGCTGTGGCCCATGGTGTGGGCCTCCCCACACGCCTCCGACA
CCACACCCTGC3′
Rhesus C1C5 mutant forward primer:
5′CTTAGCGGCCGCCACCATGTGGTGGCGCCTGTGGTGGCTGCTGCTGCT
GCTGCTGCTGCTGTGGCCCATGGTGTGGGCCTGCCCACACGCCTGCGACA
CCACACCCTGCT3′
Reverse rhesus primer:
5′CTTAGCGGCCGCTCAGCTCATCTCCAA
AGAGTTGATG3′

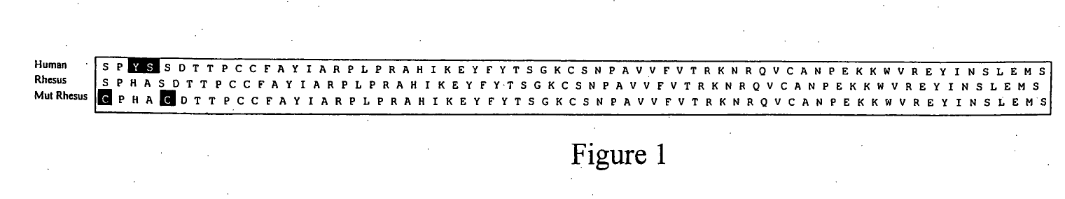

Furthermore, all primers were engineered with Not I restriction enzyme sites to facilitate molecular cloning. FIG. 1 illustrates the amino acid differences between the mature human and rhesus (wild-type and C1C5 mutant) RANTES.

The PCR products were subsequently cloned into pCR2.1 TOPO TA cloning vector (Invitrogen) and constructs containing the 300 bp rhRANTES amplification product were confirmed by DNA sequencing.

B. Cloning rhRANTES cDNA into Plasmid pTP-1/β-gal (rAAV-1 Producer Plasmid)

Upon DNA sequence confirmnation, one pCR2.1 clone each for the wild-type and C1C5 mutant were digested with Not I restriction enzyme to release the RANTES coding region which was then ligated into a Not I digested pTP-1/β-gal cloning vector. The approach was similar in concept to that previously published in Clark et al., Human Gene Therapy, 6:1329-1341 (1995). Specific modifications to construct the pTP-1/β-gal plasmid construct were as follows: First, a rAAV vector carrying the E. coli lac Z transgene was generated and was termed pAAV/CMV/β-gal. The base rAAV vector was derived from psub201 [Samulski et al., J. Virol., 61:3096-3101 (1987)], which contains a wild-type AAV genome that has been altered to contain convenient restriction enzyme sites (Xba I) that facilitate removal of the rep and cap genes and insertion of a 4.5 kb CMV promoter—E. coli lac Z—SV40 poly A transgene expression cassette (released by Pst I restriction enzyme digestion) from plasmid pCMVβ (Clontech). The resulting rAAV vector was 4.9 kb in length (including AAV2 inverted terminal repeats), which was 104.7 % of wild-type genome length.

Secondly, a plasmid DNA construct designated pBS/rep2-cap1/neotk was generated. In brief, a 4.4 kb restriction fragment containing the AAV2 rep and AAV1 cap sequences was obtained by Not I and NgoM IV restriction enzyme digestion of plasmid pXR1 (Rabinowitz et al., J. Virol., 76(2):791-801 (2001). The rep2-cap1 DNA fragment was blunt end ligated into Xba I restricted plasmid pBS/neotk vector (contains the SV40 early promoter—neomycin phosphotransferase gene-thymidine kinase polyadenylation site cassette cloned into pBluescript KS-)

Lastly, the construction of a tripartite plasmid was accomplished by removing the rep2-cap1/neo^{r} cassette from pBS/rep2-cap1/neotk (via Not I and Cla I restriction enzyme digestion) and inserting it into the unique NgoM I site in pAAV/CMV/β-gal. Thus, the pTP-1/β-gal tripartite plasmid contained: (i) a rAAV vector genome (rAAV/β-gal), (ii) rep2-cap1, and (iii) the neo^{r} gene.

Recombinant clones containing the RANTES coding regions were identified by Not I restriction and confirmed by DNA sequencing. Two correct and two reverse orientation rhRANTES (wild-type and C1C5 mutant) clones (4 total) were chosen for further analysis.

C. Cloning rhRANTES cDNA into Self-Complementary (SC) rAAV-1 Vectors

To construct the SC rAAV-I producer plasmids, the rhRANTES transgenes (the wild-type or C1C5 mutant) were cloned into a self-complementary rAAV-1 producer plasmid (pTP-1/SC-X5) using Not I restriction sites. The pTP-1/SC-X5 plasmid was made in several steps. First, the X5 scFv coding sequence (800 bp) was PCR amplified with Not I restriction site ends from plasmid pCombX/X5 scFv (gift from Dr. Dennis Burton, The Scripps Research Institute, La Jolla Calif.) and cloned into pAAV/CMV/β-gal following removal of the 3.4 kb lac Z gene by Not I restriction enzyme digestion to yield plasmid pAAV/CMV/X5. Next; the 1.8 kb CMV-X5-SV40 poly A cassette was PCR amplified with Hpa I and Xba I restriction site ends and sequence identity confirmed by sequencing. This DNA fragment was cloned into plasmid pHpa7 (gift of R. Jude Samulski, University of North Carolina) that was restricted with Hpa I and Xba I. pHpa7 plasmid contains a deletion in the 5′ AAV2 inverted terminal repeat that results in the packaging of double-stranded self-complementary vectors (McCarty et al., Gene Ther., 10(26):2112-2118 (2002). The resulting plasmid was termed pAAV/CMV/SC-X5. Lastly, the construction of a tripartite plasmid was accomplished by removing the rep2-cap1/neo^{r} cassette from pBS/rep2-cap1/neotk (via Not I and Cla I restriction enzyme digestion) and inserting it into the unique Swa I site in pAAV/CMV/SC-X5. Thus, the pTP-1/SC-X5 tripartite plasmid contained: (i) a rAAV vector genome with a mutated 5′ inverted terminal repeat (rAAV/CMV/SC-X5), (ii) rep2-cap1, and (iii) the neo^{r} gene.

Correct constructs containing the rhRANTES transgenes were confirmed by restriction enzyme digestion and DNA sequencing. Additionally, DNA sequence analysis confirmed that the SC vectors contained the expected Hpa I-Xba I deletion in the 5′ viral inverted terminal repeat (ITR).

D. Expression of rhRANTES from r/TP-1/rhRANTES Plasmids

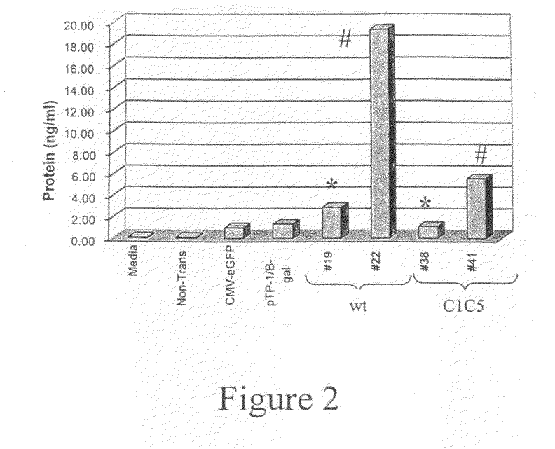

To demonstrate the pTP-1/rhRANTES vectors were functional, BHK-21 (baby hamster kidney) cells were transfected with the rhRANTES plasmid clones using Superfect transfection reagent (Qiagen Inc.). Forty-eight hours post-transfection, cell culture supernatant was analyzed for the presence of RANTES (ng/ml) using a commercial human RANTES ELISA (R&D Systems, Inc.). As seen in FIG. 2 wherein “*” denotes reverse orientation and “#” denotes correct orientation, BHK-21 cells produced significant amounts of secreted rhRANTES compared to negative control plasmid transfections (CMV-eGFP and pTP-1/β-gal). Furthermore, rAAV-1/RANTES plasmids containing the transgene in the reverse orientation produced background levels of RANTES. The amount of C1C5 rhRANTES found in the cell supernatant was consistently lower than the wild-type rhRANTES, but this is likely due to reduced antibody affinity in the commercial ELISA for the mutated C1C5 form.

E. rhRANTES Expression from SC rAAV-1/rhRANTES Plasmids

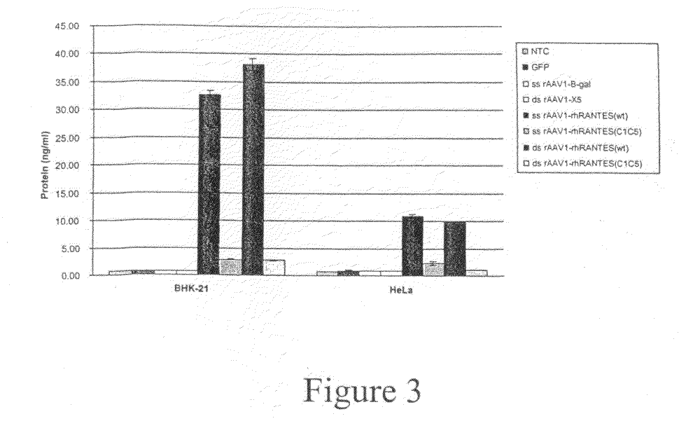

The recombinant SC plasmids were also competent for RANTES production. rhRANTES production from single-stranded (pTP-1) and double-stranded SC rAAV1/rhRANTES production plasmids were compared in BHK-21 or HeLa cells. Forty-eight hours after plasmid transfection, the supernatant was assayed by ELISA for RANTES protein expression. As shown in FIG. 3, the SC vectors produced significant amounts of secreted RANTES compared to the negative control DNA plasmid transfections (rAAV-1-β-gal and rAAV1-X5). Again, the level of wild-type RANTES production was greater than that observed for the C1C5 mutant constructs. Data are the average of 3 separate transfections.

F. Transient rAAV1/rhRANTES Vector Production (Passage Assay).

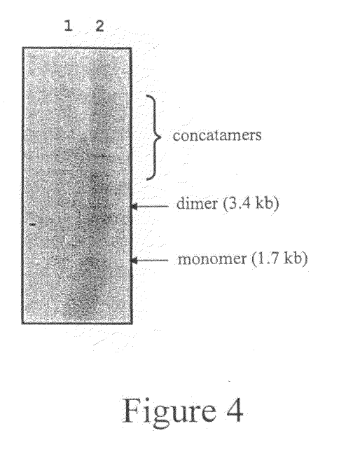

To demonstrate the rAAV producer plasmids were able to replicate and generate infectious rAAV particles efficiently, a passage assay was performed. Briefly, HeLa cell were transfected with the rAAV1/rhRANTES plasmids and subsequently infected with adenovirus type 5 (Ad5) at an moi=20. Forty-eight hours later, cells were harvested and crude cell lysates were prepared. Following heat inactivation (56° C., 30 min) of the Ad5, a 1:10 dilution of the clarified cell lysate was added to C12 cells (AAV2 rep expressing cell line) in the presence of a Ad5. Forty-eight hours later, low molecular weight DNA was extracted from the cells. Following gel electrophoresis, Southern blot DNA hybridization was performed to visualize AAV replication intermediates. As shown in FIG. 4, detectable monomeric (1.7 kb) and dimeric (3.4 kb) replication forms were observed in the C12 DNA indicative of infectious rAAV1 formation in the HeLa cell clarified lysate. In FIG. 4, Lane 1 is pTP-1/wt rhRANTES+Ad5 and Lane 2 is pTP-1/C1C5 rhRANTES+Ad5. Hybridizing DNA fragments were detected that corresponded to the expected sizes of replication competent virus: momomeric form=1.7 kb, dimeric form=3.4 kb. Replication forms were present in neither HeLa cells infected with Ad5 nor HeLa cells transduced with cell lysates prepared from pTP-1/rhRANTES transfected HeLa cells minus Ad5 infection.

Similar results were obtained for the SC rAAV1/rhRANTES recombinant vectors, confirming the ability of the plasmid constructs to generate infectious rAAV1/rhRANTES particles (data not shown).

G. rAAV1/rhRANTES Particles are Infectious and Produce RANTES Following Transduction of Cells in Culture.

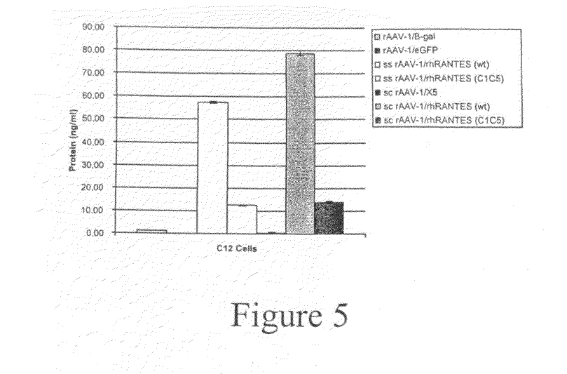

To document the ability of the rAAV1/rhRANTES viral vectors to mediate secreted RANTES expression following infection of cells in culture, a small-scale viral preparation (via transient plasmid transfection) was generated and used to infect C12 cells (moi=1,000 DNase resistant particles per cell) in absence or presence of Ad5 (moi=20). RANTES ELISA was performed on the cell culture supernatants from C12 cells transduced with rAAV1/rhRANTES vectors (wild-type and C1C5 mutant) or self-complementary derivatives (wild-type and C1C5 mutant). As shown in FIG. 5, all 4 rAAV1/rhRANTES vectors (ss wild-type, ss C1C5, SC wild-type, and SC C1C5) produced significantly greater amounts of RANTES compared to rAAV1/vector negative controls (rAAV1/β-gal, rAAV1/GFP, and SC rAAV1/X5). Consistent with the plasmid transfection data, the wild-type vectors appear to produce greater levels of RANTES compared to the C1C5 mutant vectors.

Similar levels and patterns of expression were observed in HeLa and BHK-21 cells after of rAAV1/rhRANTES vector infection (data not shown).

H. Construction of Stable rAAV1 Producer Cell Lines.

To facilitate large-scale viral production, HeLa-based cell lines were constructed by plasmid DNA transfection and drug resistance selection using the four pTP-1/rhRANTES plasmids (ss wild-type, ss C1C5 mutant, SC wild-type, and SC C1C5 mutant). Optimal producer cell lines were selected essentially as described by Clark et al., Hum. Gene Ther., 6: 1329-1341 (1995). Positive cell lines containing a replicating rAAV genome were expanded and the DNase resistant particles (DRP) per cell productivity determined by quantitative Taqman PCR analysis. Table 1 shows the DRP per cell values for the clones that were subsequently sent to the viral vector core for cell-cube production.

TABLE 1
Optimal HeLa-based rAAV1/rhRANTES producer cell lines.

| Vector Produced | Vector Yield (DRP/cell) |
|---|---|
| rAAV1/rhRANTES (wild-type) | 10,200 |
| rAAV1/rhRANTES (C1C5 Mutant) | 3,500 |
| SC rAAV1/rhRANTES (wild-type) | 3,100 |
| SC rAAV1/rhRANTES (C1C5 mutant) | 9,000 |

### EXAMPLE 2

### Delivery and Expression of Genes Encoding HIV-1 Fusion Inhibitor Peptides T-20 and T-1249 to Inhibit HIV-1 Replication and Growth

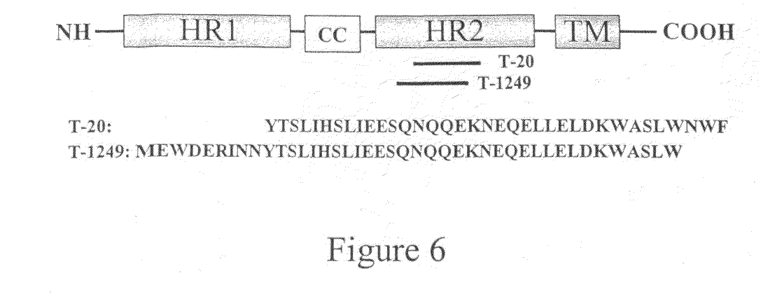

A second attractive target for HIV-1 entry inhibition is the final step of the HIV-1 infection process, fusion of the viral envelope with the cell membrane. Fusion is mediated by the gp41 envelope glycoprotein and a model of gp41-mediated membrane fusion analogous to the “spring-loaded” mechanism of influenza virus has been proposed. The sequence of gp41 contains two heptad-repeat regions termed HR1 and HR2 that denote the presence of hydrophobic regions found in 2 alpha-helical “coiled-coil” structures. Significantly, mutations in these HR regions interfere with the fusion property of gp41. The model predicts that the gp120-gp41 trimer holds each gp41 moiety in a high-energy configuration, with the fusion peptide pointed inward, toward the viral surface. The binding of gp120 to CD4 and chemokine co-receptors appears to release gp41 from this configuration, causing the fusion peptide to “spring” outward toward the cell membrane. The HR1 regions then fold over into the hydrophobic groove formed by the three corresponding HR2 regions, forming a stable six-helix bundle, resulting in the juxtaposition of viral and cellular membranes and ultimately fusion. Two gp41 HR2 peptides T-20 and T-1249 are currently being studied as small molecule inhibitors of HIV-1 fusion. T-20 and T-1249 partially overlap, but T-1249 extends into a “deep-pocket” region of HR1 that is important for the formation of the six-helix structure required for fusion (FIG. 6). These competitive inhibitors are thought to bind to the HR1 region and “lock” it into a non-fusogenic conformation. Both peptides appear to possess broad activity against X4, R5, and dual tropic variants of HIV-1. Importantly, oral treatment with this large peptide is not feasible and daily intravenous doses of peptide are required for therapeutic effect.

The present inventors contemplate that therapeutic levels of circulating T-20/T-1249 can be achieved via rAAV mediated muscle-targeted gene transfer. Towards this objective, rAAV-1 based vectors expressing the T-20 or T-1249 peptides have been constructed as described below. In order to maximize circulating T-20 and T-1249 expression levels, constructs were first engineered to include an optimized synthetic leader sequence to increase the efficiency of protein secretion from muscle cells into the systemic circulation. Second, the T-20 DNA was synthesized by Retrogen Inc. using optimal human codon usage to enhance gene expression. Third, to maximize gene transfer levels into myocytes, rAAV-1 serotype vectors were constructed. Fourth, self-complementary rAAV/chemokine vectors were generated.

### A. T-20 DNA Synthesis and Genome Generation

A synthetic, codon-optimized T-20 oligonucleotide was generated by Retrogen Inc. The sequence generated was as follows:

5′gcggccgccaccATGTGGTGGCGCCTGTGGTGGCTGCTGCTGCTGCTG
CTGCTGCTGTGGCCCATGGTGTGGGCCTACACCTCCCTGATCCACTCCCT
GATCGAGGAGTCCCAGAACCAGCAGGAGAAGAACGAGCAGGAGCTGCTGG
AGCTGGACAAGTGGGCCTCCCTGTGGAACTGGTTCTGAGcggccgc3′.

The gene possesses flanking Not I restriction sites (lower case letters) to facilitate cloning and a 5′ Kozak consensus sequence (ccacc) for efficient translation initiation. Additionally, the construct encodes a 21 amino acid synthetic secretory leader sequence that provides increased secretion.

The T-20 gene was cloned into (via Not I restriction sites) the rAAV-1 producer plasmid pTP-1/β-gal to yield pTP-1/SL-T20 and recombinant clones confirmed by DNA sequencing. Similarly, the T-20 gene was cloned into the SC rAAV-1 producer plasmid (pTP-1/SC-X5) using Not I restriction sites to generate the hairpin vector (pTP-1/SC/SL-T20).

### B. T-1249 Genome Constructions

Taking advantage of significant sequence overlap between the T-20 and T-1249 sequences, PCR amplification was used to generate the T-1249 gene using the T-20 gene as the PCR template.

Producer plasmids were generated that included the optimized synthetic leader sequence or the native leader sequences one of two highly secreted proteins, cystatin and alpha-1 anti-trypsin. Three forward PCR primers were synthesized to incorporate the appropriate leader sequence and add the 9 additional N-terminal amino acids to the T-20 template sequence. The primer sequences were:

(i) artificial signal peptide
5′ATTCAGCGGCCGCCACCATGTGGTGGCGCCTGTGGTGGCTGCTGCTGC
TGCTGCTGCTGCTGTGGCCCATGGTGTGGGCCATGGAGTGGGACAGGGAG
ATCAACAACTAC3′
(ii) cystatin leader peptide,
5′ATTCAGCGGCCGCCACCATGGCCCGCCCCCTGTGCACCCTGCTGCTGC
TGATGGCCACCCTGGCCGGCGCCCTGGCCATGGAGTGGGACAGGGAGATC
AACAACTAC3′
(iii) A1AT leader peptide
5′ATTCAGCGGCCGCCACCATGCCCTCCTCCGTGTCCTGGGGCATCCTGC
TGCTGGCCGGCCTGTGCTGCCTGGTGCCTGTGTCCCTGGCCATGGAGTGG
GACAGGGAGATCAACAACTAC3′
(iv) reverse T-1249 primer
5′ATTCAGCGGCCGCCTCACCACAGGGA GGCCCACTTGTCC3′

The three PCR products were cloned into pTP-1/β-gal as described above using Not I restriction sites.

### C. T-1249 Secretion in Cell Culture

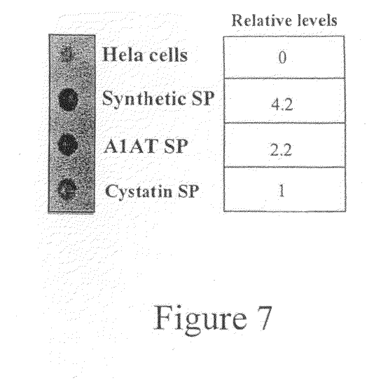

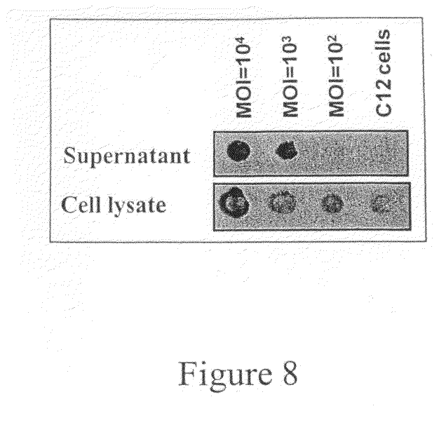

To compare the efficiency of T-1249 secretion into cell culture media, HeLa cells were transfected (Superfect; Qiagen Inc.) with the pTP-1/T-1249 plasmids and T-1249 levels quantified in cell culture supernatants (1:5 dilution) 48 hr post-transfection. Levels were determined by extrapolation from a standard curve generated using a T-20 peptide standard and an HIV-1 neutralizing monoclonal antibody (2F5) that recognizes a linear epitope (ELDKWA) present within both peptides. Western dot blot assay sensitivity was determined to be 20 ng T-20/dot. To normalize for transfection efficiency, a second plasmid encoding the E. coli lacZ gene was included in the transfection (pCMV/β-gal) and β-galactosidase levels quantified using a commercial kit (All-in-One β-gal kit, Pierce Chemical). Normalization for the transfection efficiency allowed for comparison of relative T-1249 levels. The experiment was performed in duplicate and data are the average of the 2 experiments. As seen in FIG. 7, the synthetic leader was approximately 2-4 fold better at mediating peptide secretion compared to the native cellular leader peptides.

D. Production of Infectious rAAV-1 Particles and Transduction of Cells in Culture

A viral passage assay was then performed to confirm the ability of plasmids pTP-1/SL-T20 and pTP-1/SL-T1249 to generate infectious rAAV1 particles, similar to that described for the RANTES vectors. Optimal rAAV-1 HeLa producer cell lines were then isolated and productivity assessed using quantitative Taqman PCR (3.6×10⁴ DRP/ml, T-20 and 1.7×10⁴ DRP/ml, T-1249). A small-scale rAAV1/SC/SL-T20 vector stock was generated by wild-type Ad5 infection (moi=20) and virus purified by iodixanol gradient fractionation and anion-exchange chromatography. A purified rAAV1/SL-T20 vector was used to infected HeLa cells (2×10⁶ cells) at the MOI indicated in FIG. 8. Forty-eight hr post-transduction cell culture supernatant was collected and a cell lysate was generated by detergent lysis. Both the supernatant (1:5 dilution of 0.2 ml) and cell lysate (1×10⁵ cell equivalents) were blotted onto a nitrocellulose membrane and T-20 levels visualized using the human 2F5 antibody (1:1,000 dilution) as the primary antibody.

As seen in FIG. 8, a dose response relationship was observed at various rAAV-1/T20 inputs (moi=1,000 DRP or 10,000 DRP), with robust T-20 secretion into the cell culture supernatant.

While the present invention has been described in terms of specific embodiments, it is understood that variations and modifications will occur to those skilled in the art. Accordingly, only such limitations as appear in the claims should be placed on the invention.

We claim:

1. A recombinant adeno-associated virus (AAV) genome comprising AAV inverted terminal repeats flanking a gene cassette of DNA encoding one or more virus entry inhibitor proteins operatively linked to transcriptional control DNA, wherein the genome lacks AAV rep and cap DNA.

2. The genome of claim 1 wherein the virus entry inhibitor protein inhibits entry of HIV, Hepatitis B virus, Hepatitis C virus, Epstein Barr Virus, influenza virus or Respiratory Syncytial Virus.

3. The genome of claim 2 wherein the virus entry inhibitor protein inhibits entry of HIV.

4. The genome of claim 3 wherein the virus entry inhibitor protein is T20, T1249, T649, 5-helix, CD4, CCR5, CXCR4, RANTES, or SDF-1.

5. An infectious encapsidated rAAV particle (rAAV) comprising a rAAV genome of claim 1.

6. A packaging cell producing a rAAV of claim 5.

7. A composition comprising one or more rAAV according to claim 5.

8. The rAAV rAAV1/CMV/T20, rAAV1/CMV/T-1249, rAAV1/CMV/RANTES, rAAV1/CMV/rhRANTES(wt) or rAAV1/CMV/mRANTES (C1C5).

9. A composition comprising one or more rAAV of claim 8.

10. A method of delivering a virus entry inhibitor protein to an animal in need thereof, comprising the step of transducing a tissue of the animal with a composition according to claim 7.
